(19) United States
(12) Patent Application Publication
(10) Pub. No.: US 2007/0005053 A1
(43) Pub. Date: Jan. 4, 2007

(54) Ablation catheter with contoured openings in insulated electrodes

(76) Inventor: Jeremy D. Dando, Plymouth, MN (US)

Correspondence Address: HEIMBECHER & ASSOC., LLC, P O BOX 33, HAMEL, MN, 55340-0033, US

(21) Appl. No.: 11/172,647

(22) Filed: Jun. 30, 2005

(51) Int. Cl. A61B 18/14

(52) U.S. Cl. 606/41

(57) ABSTRACT

An array of ring electrodes or a wire electrode is mounted about the outside surface of the distal end of the ablation catheter. Substantially all of the outer surface of each ring or wire electrode is covered by an electrically insulating coating. The insulating surface coating on each ring defines a contoured opening in the insulating surface coating that exposes the conductive band or wire beneath. An array of contoured openings are formed along a wire electrode. The insulating coating mitigates potential edge effects that create hot spots and can result in unwanted tissue damage during an ablation procedure.

### BACKGROUND OF THE INVENTION

a. Field of the Invention

The instant invention is directed to the field of intravasuclar catheters for ablation of tissue. In particular, the invention relates to forms of ring electrodes positioned at a distal end of a catheter to perform an ablation procedure.

b. Background Art

A catheter is generally a very small diameter tube for insertion into the body for the performance of medical procedures. Among other uses, catheters can be used to examine, diagnose, and treat disease while positioned at a specific location within the body that is otherwise inaccessible without more invasive procedures. During these procedures a catheter is inserted into the patient's vasculature near the surface of the body and is guided to a specific location within the body for examination, diagnosis, and treatment. For example, one procedure utilizes a catheter to convey an electrical stimulus to a selected location within the human body. Another procedure utilizes a catheter with sensing electrodes to monitor various forms of electrical activity in the human body.

In a normal heart, contraction and relaxation of the heart muscle (myocardium) takes place in an organized fashion as electrochemical signals pass sequentially through the myocardium from the sinoatrial (SA) node located in the right atrium, to the atrialventricular (AV) node in the septum between the right atrium and right ventricle, and then along a well-defined route which includes the His-Purkinje system into the left and right ventricles. Sometimes abnormal rhythms occur in the atria that are referred to as atrial arrhythmia. Three of the most common arrhythmia are ectopic atrial tachycardia, atrial fibrillation, and atrial flutter. Arrhythmia can result in significant patient discomfort and even death because of a number of associated problems, including the following: (1) an irregular heart rate, which causes a patient discomfort and anxiety; (2) loss of synchronous atrioventricular contractions, which compromises cardiac hemodynamics resulting in varying levels of congestive heart failure; and (3) stasis of blood flow, which increases the vulnerability to thromboembolism.

It is sometimes difficult to isolate a specific pathological cause for the arrhythmia, although it is believed that the principal mechanism is one or a multitude of stray circuits within the left and/or right atrium. These circuits or stray electrical signals are believed to interfere with the normal electrochemical signals passing from the SA node to the AV node and into the ventricles. Efforts to alleviate these problems in the past have included significant usage of various drugs. In some circumstances drug therapy is ineffective and frequently is plagued with side effects such as dizziness, nausea, vision problems, and other difficulties.

An increasingly common medical procedure for the treatment of certain types of atrial arrhythmia and other cardiac arrhythmia involves the ablation of tissue in the heart to cut-off the path for stray or improper electrical signals. The particular area for ablation depends on the type of underlying arrhythmia. Originally, such procedures actually involved making incisions in the myocardium (hence the term “ablate,” which means to cut) to create scar tissue that blocked the electrical signals. These procedures are now often performed with an ablation catheter.

Ablation catheters do not physically cut the tissue. Instead they are designed to apply electrical energy to areas of the myocardial tissue causing tissue necrosis by coagulating the blood supply in the tissue and thus halt new blood flow to the tissue area. The necrosis lesion produced electrically isolates or renders the tissue non-contractile. The lesion partially or completely blocks the stray electrical signals to lessen or eliminate arrhythmia. Typically, the ablation catheter is inserted into an artery or vein in the leg, neck, or arm of the patient and threaded, sometimes with the aid of a guide wire or introducer, through the vessels until a distal tip of the ablation catheter reaches the desired location for the ablation procedure in the heart.

It is well known that benefits may be gained by forming lesions in tissue if the depth and location of the lesions being formed can be controlled. In particular, it can be desirable to elevate tissue temperature to around 50° C. until lesions are formed via coagulation necrosis, which changes the electrical properties of the tissue. For example, when sufficiently deep lesions are formed at specific locations in cardiac tissue via coagulation necrosis, undesirable ventricular tachycardias and atrial flutter may be lessened or eliminated. “Sufficiently deep” lesions means transmural lesions in some cardiac applications.

It has been discovered that more effective results may be achieved if a linear lesion of cardiac tissue is formed. The term “linear lesion” as used herein means an elongate, continuous lesion, whether straight or curved, that blocks electrical conduction. The ablation catheters commonly used to perform these procedures produce electrically inactive or noncontractile tissue at a selected location by physical contact of the cardiac tissue with an electrode of the ablation catheter. Current techniques for creating continuous linear lesions in endocardial applications include, for example, dragging a conventional catheter on the tissue, using an array electrode, or using pre-formed curved electrodes. Curved electrodes have also been formed by guiding a catheter with an array electrode over a wire rail The wire rail is formed as a loop, thus guiding the distal end of the catheter into a loop form as well. The array electrodes and curved electrodes are generally placed along the length of tissue to be treated and energized to create a lesion in the tissue contiguous with the span of electrodes along the curved or looped surface. Alternately, some catheter designs incorporate steering mechanisms to direct an electrode at the distal tip of the catheter. The clinician places the distal tip electrode of the catheter on a targeted area of tissue by sensitive steering mechanisms and then relocates the electrode tip to an adjacent tissue location in order to form a continuous lesion.

During conventional ablation procedures, the ablating energy is delivered directly to the cardiac tissue by an electrode on the catheter placed against the surface of the tissue to raise the temperature of the tissue to be ablated. Care must be taken to prevent the excessive application of energy, which can result in tissue damage beyond mere necrosis and instead actually decompose, i.e., char, the tissue. Such excessive tissue damage can ultimately weaken and compromise the myocardium. The rise in tissue temperature also causes a rise in the temperature of blood surrounding the electrode. This often results in the formation of coagulum on the electrode, which reduces the efficiency of the ablation electrode. With direct contact between the electrode and the blood, some of the energy targeted for the tissue ablation is dissipated into the blood. This coagulation problem can be especially significant when linear ablation lesions or tracks are produced because such linear ablation procedures conventionally take more time than ablation procedures ablating only a single location.

The information included in this background section of the specification, including any references cited herein and any description or discussion thereof, is included for technical reference purposes only and is not to be regarded subject matter by which the scope of the invention is to be bound.

### BRIEF SUMMARY OF THE INVENTION

The present invention is directed to an improved design for ring or wire electrode ablation catheters used, for example, in cardiac ablation procedures to produce lesions in cardiac tissue. The ring or wire electrodes are mounted on the outside surface of the distal end of the ablation catheter in order to be placed into contact with the target tissue. In the present invention, substantially all of the outer surface of each ring or the wire electrode is covered by an electrically insulating coating. The insulating surface coating on each ring electrode or the wire electrode defines a contoured opening in the insulating surface coating that exposes the conductive electrode beneath. In a series of ring electrodes or along a single helical wire electrode, each of the contoured openings is positioned in a linear array parallel to the longitudinal direction of the catheter.

In one form of the invention, a catheter comprises an elongate shaft defining a lumen extending distally from a proximal section. At least one electrode is positioned about a distal end of the elongate shaft. The at least one electrode further comprises a conductive material and an insulating coating substantially covering the conductive material. The insulating coating defines a contoured opening that exposes an area of the conductive material. At least one electrode lead is housed within the lumen, extends from the proximal section, and is coupled at a distal end with the at least one electrode.

In another form of the invention, a catheter comprises an elongate shaft defining a lumen extending distally from a proximal section. A plurality of electrode rings is positioned about a distal end of the elongate shaft. Each of the plurality of electrode rings encircles a respective portion of the elongate shaft and is spaced apart from each adjacent electrode ring by a uniform distance. Each of the plurality of electrode rings further comprises a conductive material and an insulating coating substantially covering the conductive material. The insulating coating defines a contoured opening that exposes an area of the conductive material. The contoured openings of each of the plurality of electrode rings are arranged longitudinally along the distal end of the elongate shaft to form a linear array. At least one electrode lead is housed within the lumen, extends from the proximal section, and is coupled at a distal end with the plurality of electrode rings.

In a further form of the invention, a catheter comprises an elongate shaft defining a lumen extending from a proximal section. A helical wire electrode is wrapped about a distal end of the elongate shaft. The helical wire electrode further comprises a conductive material and an insulating coating substantially covering the conductive material. The insulating coating defines a plurality of contoured openings that each expose an area of the conductive material. Each of the plurality of contoured openings is positioned circumferentially about the elongate shaft in-line with each adjacent contoured opening to form a linear array parallel to the longitude of the elongate shaft. Each turn of the helical electrode wire is spaced sufficiently close to each adjacent turn at a regular, narrow interval to provide sufficient energy overlap to produce a linear lesion correlative to a length of the helical wire electrode. At least one electrode lead is housed within the lumen, extends from the proximal section, and is coupled at a distal end with the helical electrode wire.

An alternative form of the invention is directed to an electrode for use in conjunction with a cardiac ablation catheter. The electrode comprises a conductive band sized to encircle an outer surface of the catheter. An insulating coating substantially covers an outer surface of the conductive band. The insulating coating defines a contoured aperture exposing a portion of the conductive band. A lead wire is electrically coupled with the conductive band.

An additional form of the invention concerns a method for minimizing variations in power density in a surface electrode positioned on a catheter. A conductive material portion of the surface electrode is coated with a biocompatible, electrically insulating coating. Then a contoured aperture is formed within the electrically insulating coating to expose an area of the conductive material portion.

Other features, details, utilities, and advantages of the present invention will be apparent from the following more particular written description of various forms of the invention as further illustrated in the accompanying drawings and defined in the appended claims.

### BRIEF DESCRIPTION OF THE DRAWINGS

FIG. 1 is an isometric view of a ablation catheter/introducer assembly including a ring electrode section according to a first embodiment of the present invention.

FIG. 2 is an elevation view of a distal portion of the catheter of FIG. 1 including the ring electrode section.

FIG. 3 is a top plan view of the catheter of FIG. 2.

FIG. 4 is an isometric view of the distal end of the catheter of FIG. 2.

FIG. 5 is a cross-section view of the catheter of FIG. 2 taken along line 5-5 as indicated in FIG. 4.

FIG. 6 is a cross-section view of the catheter of FIG. 2 taken along line 6-6 as indicated in FIG. 5, wherein separate electrode leads are coupled with each ring electrode.

FIG. 7 is a cross-section view the distal end of a catheter (similar to FIG. 6) according to a second embodiment of the invention, wherein a single electrode lead is coupled with each of the ring electrodes.

FIG. 8 is an isometric view of the distal end of a catheter according to a third embodiment of the invention incorporating a single coil electrode in lieu of separate ring electrodes.

FIG. 9 is an enlarged plan view of one of the ring electrodes with a contoured opening according to a fourth embodiment of the present invention.

FIG. 10 is an enlarged plan view of one of the ring electrodes with a contoured opening according to a fifth embodiment of the present invention.

FIG. 11 is an enlarged plan view of one of the ring electrodes with a contoured opening according to a sixth embodiment of the present invention.

FIG. 12 is an enlarged plan view of one of the ring electrodes with a contoured opening according to a seventh embodiment of the present invention.

FIG. 13 is an enlarged plan view of one of the ring electrodes with a contoured opening according to a eighth embodiment of the present invention.

FIG. 14 is an enlarged plan view of one of the ring electrodes with a contoured opening according to a ninth embodiment of the present invention.

FIG. 15 is an enlarged plan view of one of the ring electrodes with a contoured opening according to a tenth embodiment of the present invention.

FIG. 16 is an enlarged plan view of one of the ring electrodes with a contoured opening according to a eleventh embodiment of the present invention.

FIG. 17 is an isometric view of a heart with portions of the atria and ventricles cut-away to reveal positioning of a generic version of the catheter of the present invention in the left atrium, adjacent to the left superior pulmonary vein.

### DETAILED DESCRIPTION OF THE INVENTION

The present invention concerns an improved design for ring or wire electrode ablation catheters used, for example, in cardiac ablation procedures to produce lesions in cardiac tissue. The ring or wire electrodes are mounted on the outside surface of the distal end of the ablation catheter in order to be placed into contact with the target tissue. In the present invention, substantially all of the outer surface of each ring or wire electrode is covered by an electrically insulating coating. The insulating coating on each ring or wire electrode defines a contoured opening in the insulating coating that exposes the conductive electrode beneath. In a series of ring electrodes or along a single helical wire electrode, each of the contoured openings is positioned in a linear array parallel to the length of the catheter.

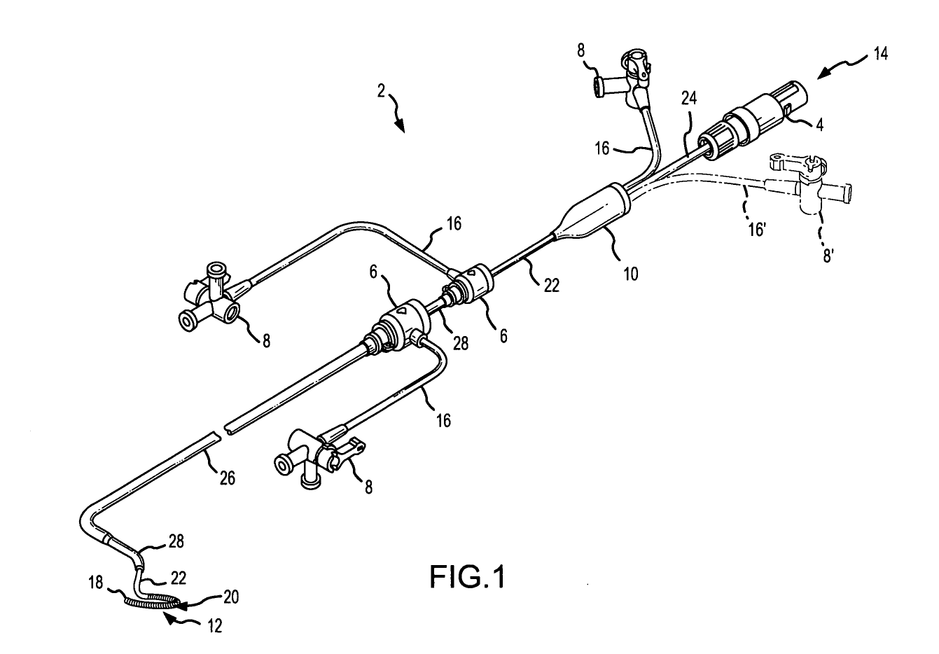

FIG. 1 is an isometric view of a catheter/introducer assembly 2 for use in conjunction with the present invention. According to a first embodiment of the present invention, a catheter 22 in the form of an elongate shaft has an electrical connector 4 at a proximal end 14 and an ablation electrode section 20, at a distal end 12. The catheter 22 is used in combination with an inner guiding introducer 28 and an outer guiding introducer 26 to facilitate formation of lesions on tissue, for example, cardiovascular tissue. The inner guiding introducer 28 is longer than and is inserted within the lumen of the outer guiding introducer 26. Alternatively, a single guiding introducer or a precurved transeptal sheath may be used instead of both the inner guiding introducer 28 and the outer guiding introducer 26. In general, introducers or precurved sheaths are shaped to facilitate placement of the ablation electrode section 20 at the tissue surface to be ablated. As depicted in FIG. 1, for example, the outer guiding introducer 26 may be formed with a curve at the distal end 12. Similarly, the inner guiding introducer 28 may be formed with a curve at the distal end 12. Together, the curves in the guiding introducers 26, 28 help orient the catheter 22 as it emerges from the inner guiding introducer 26 in a cardiac cavity. Thus, the inner guiding introducer 28 and the outer guiding introducer 26 are used navigate a patient's vasculature to the heart and through its complex physiology to reach specific tissue to be ablated. The guiding introducers 26, 28 need not be curved or curved in the manner depicted depending upon the desired application.

As shown in FIG. 1, each of the guiding introducers 26, 28 is connected with a hemostatic valve 6 at its proximal end to prevent blood or other fluid that fills the guiding introducers 26, 28 from leaking before the insertion of the catheter 22. The hemostatic valves 6 form tight seals around the shafts of the guiding introducers 26, 28 or the catheter 22 when inserted therein. Each hemostatic valve 6 may be have a port connected with a length of tubing 16 to a fluid introduction valve 8. The fluid introduction valves 8 may be connected with a fluid source, for example, saline or a drug, to easily introduce the fluid into the introducers, for example, to flush the introducer or to inject a drug in to the patient. Each of the fluid introduction valves 8 may control the flow of fluid into the hemostatic valves 16 and thereby the guiding introducers 26, 28.

The proximal end 14 of the catheter 22 may include a catheter boot 10 that seals around several components to allow the introduction of fluids and control mechanisms into the catheter 22. For example, at least one fluid introduction valve 8 with an attached length of tubing 16 may be coupled with the catheter boot 10. An optional fluid introduction valve 8′ and correlative tube 16′ (shown in phantom) may also be coupled with the catheter boot 10, for example, for the introduction of fluid into a catheter with multiple fluid lumens if separate control of the pressure and flow of fluid in the separate lumens is desired. The electrical connector 4 for connection with a control handle, an energy generator, and/or sensing equipment (none shown) may be coupled with the catheter boot 10 via a control shaft 24. The control shaft 24 may enclose, for example, control wires for manipulating the catheter 22 or ablation electrode section 20, conductors for energizing an electrode in the ablation electrode section 20, and/or lead wires for connecting with sensors in the ablation electrode section 20. The catheter boot 10 provides a sealed interface to shield the connections between such wires and fluid sources and one or more lumen in the catheter 22 through which they extend.

The catheter may be constructed from a number of different polymers, for example, polypropylene, oriented polypropylene, polyethylene, polyethylene terephthalate, crystallized polyethylene terephthalate, polyester, polyvinyl chloride (PVC), polytetraflouroethylene (PTFE), expanded polytetraflouroethylene (ePTFE), and Pellethane®. Alternatively, the catheter 22 may be composed, for example, of any of several formulations of Pebax® resins (AUTOFINA Chemicals, Inc., Philadelphia, Pa.), or other polyether-block co-polyamide polymers. By using different formulations of the Pebax® resins for different sections of the catheter, different material and mechanical properties, for example, flexibility or stiffness, can be chosen for different sections along the length of the catheter.

The catheter may also be a braided catheter wherein the catheter wall includes a cylindrical and/or flat braid of metal fibers (not shown), for example, stainless steel fibers. Such a metallic braid may be included in the catheter to add stability to the catheter and also to resist radial forces that might crush the catheter. Metallic braid also provides a framework to translate torsional forces imparted by the clinician on the proximal end 12 of the catheter 22 to the distal end 12 to rotate the catheter 22 for appropriate orientation of the ablation electrode section 20.

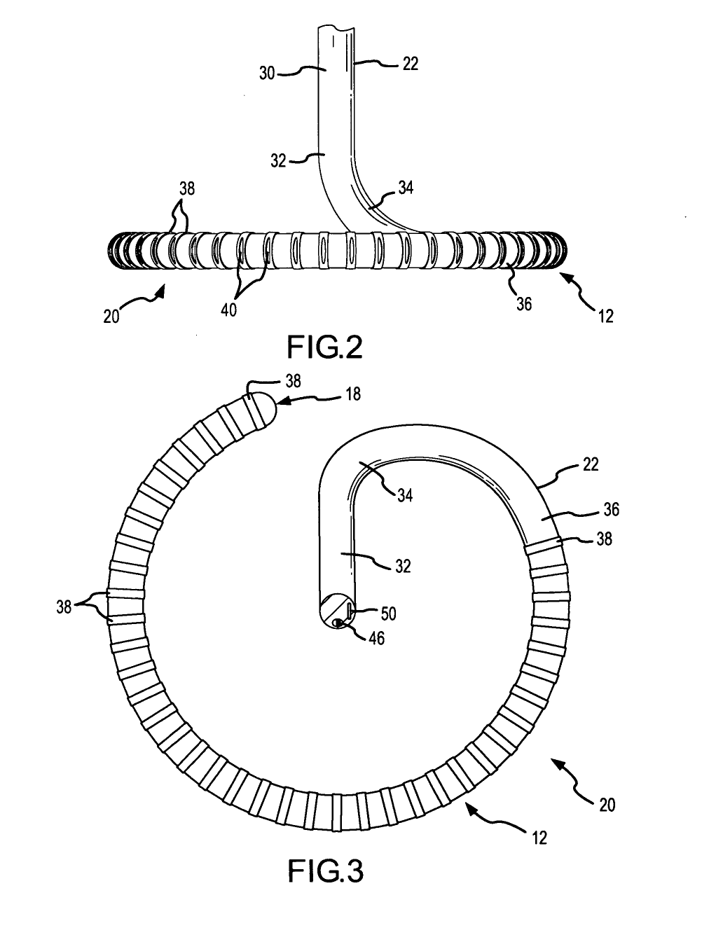

The distal end of the catheter may be straight or take on a myriad of shapes depending upon the desired application. The distal end 12 of one embodiment of a catheter 22 according to the present invention is shown in greater detail in FIGS. 2 and 3. In the embodiment shown in FIGS. 2 and 3, the catheter 22 consists mainly of a “straight” section 30 extending from the catheter boot 10 at the proximal end 14 to a point adjacent to the distal end 12 of the catheter/introducer assembly 2 (see the exemplary catheter of FIG. 1). The straight section 30 is generally the portion of the catheter 22 that remains within the vasculature of the patient while a sensing or ablation procedure is performed by a clinician. At the distal end 12, the catheter 22 is composed of a first curved section 32 and a second curved section 34 before transitioning into a third curved section 36 that forms the ablation electrode section 20. The first curved section 32 is adjacent and distal to the straight section 30 and proximal and adjacent to the second curved section 34. The second curved section 34 is itself proximal and adjacent to the third curved section 36.

The straight section 30, first curved section 32, second curved section 34, and third curved section 36 may together form a single, unitary structure of the catheter 22, but may originally be separate pieces joined together to form the catheter 22. For example, as indicated above, each of the different sections of the catheter may be composed of different formulations of Pebax® resins, or other polyether-block co-polyamide polymers, which can be used to create desired material stiffness within the different sections of the catheter. By joining separate curved sections or unitarily molding the distal end of the catheter shaft 22 proximal to the ablation electrode section 20 using a relatively stiff resin, a desired shape can be imparted to that section of the catheter shaft 22 to effect the ultimate orientation of the ablation electrode section 20.

As shown in FIGS. 2 and 3, the first curved section 32 and second curved section 34 of the catheter 22 align the third curved section 36 such that it is transverse to the orientation of the straight section 30 of the catheter 22. The ablation electrode section 20 assumes the shape of the third curved section 36 and forms a generally C-shaped or lasso-like configuration when deployed from the inner guiding introducer 28. In addition, the distal end of the straight section 30 of the catheter 22 is oriented in a position where a longitudinal axis extending through the distal end of the straight section 30 passes orthogonally through the center of a circle defined by the C-shaped third curved section 36. In this manner the straight section 30 of the catheter 22 is spatially displaced from the ablation electrode section 20 so that the straight section 30 is unlikely to interfere with the interface between the ablation electrode section 20 extending along the third curved section 36 and the cardiac tissue as further described below.

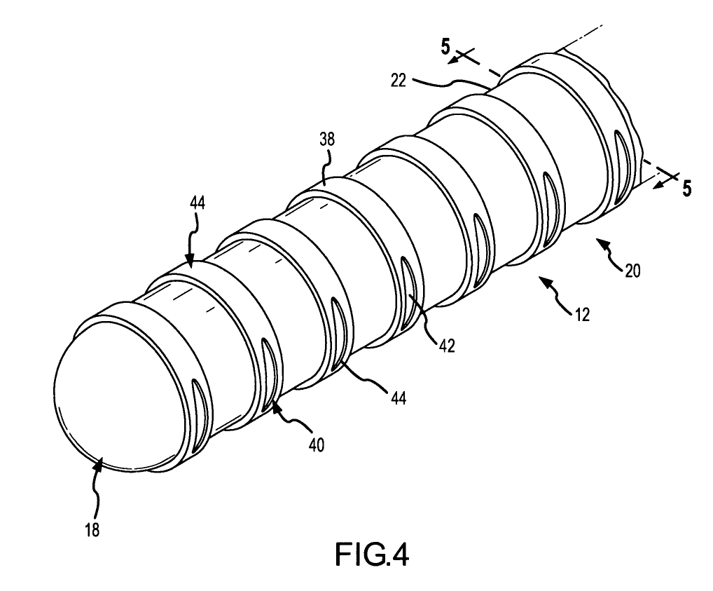

The catheter 22 may further house a shape-retention or shape-memory wire 50 in order to impart a desired shape to the distal end 12 of the catheter 22 in the area of the ablation electrode section 20. See also FIGS. 5-7. A shape-retention or shape-memory wire 50 is flexible while a clinician negotiates the catheter 22 through the vasculature to reach the heart and enter an atrial chamber. Once the distal end 12 of the catheter 22 reaches the desired cardiac cavity with the ablation electrode section 20, the shape-retention/shape-memory wire 50 can be caused to assume a pre-formed shape form, e.g., the C-shaped configuration of the ablation electrode section 20, to accurately orient the ablation electrode section 20 within the cardiac cavity for the procedure to be performed. The C-shaped configuration of the ablation electrode section 20 as shown in FIGS. 2 and 3 may be imparted to the catheter through the use of such shape-retention or shape-memory wires, in addition to or in lieu of pre-molding of the catheter material, to appropriately conform to tissue or to the shape of a cavity in order to create the desired lesion at a desired location.

In one embodiment, the shape-retention/shape-memory wire 50 may be NiTinol wire, a nickel-titanium (NiTi) alloy, chosen for its exceptional shape-retention/shape-memory properties. When used for shape-memory applications, metals such as NiTinol are materials that have been plastically deformed to a desired shape before use. Then upon heat application, either from the body as the catheter is inserted into the vasculature or from external sources, the shape-memory material is caused to assume its original shape before being plastically deformed. A shape-memory wire generally exhibits increased tensile strength once the transformation to the pre-formed shape is completed. NiTinol and other shape-memory alloys are able to undergo a “martenistic” phase transformation that enables them to change from a “temporary” shape to a “parent” shape at temperatures above a transition temperature. Below the transition temperature, the alloy can be bent into various shapes. Holding a sample in position in a particular parent shape while heating it to a high temperature programs the alloy to remember the parent shape. Upon cooling, the alloy adopts any temporary shape imparted to it, but when heated again above the transition temperature, the alloy automatically reverts to its parent shape

Common formulas of NiTinol have transformation temperatures ranging between −100 and +110° C., have great shape-memory strain, are thermally stable, and have excellent corrosion resistance, which make NiTinol exemplary for use in medical devices for insertion into a patient. For example, the shape-memory wire may be designed using NiTinol with a transition temperature around or below room temperature. Before use the catheter is stored in a low-temperature state. By flushing the fluid lumen with chilled saline solution, the NiTinol shape-memory wire can be kept in the deformed state while positioning the catheter at the desired site. When appropriately positioned, the flow of chilled saline solution can be stopped and the catheter, either warmed by body heat or by the introduction of warm saline, promotes recovery by the shape-memory wire to assume its “preprogrammed” shape, forming, for example, the C-shaped curve of the ablation electrode section.

Alternately, or in addition, shape-memory materials such as NiTinol may also be super elastic—able to sustain a large deformation at a constant temperature—and when the deforming force is released they return to their original, undeformed shape. Thus the catheter 22 incorporating NiTinol shape-retention wire 50 may be inserted into the generally straight lengths of introducer sheaths to reach a desired location and upon emerging from the introducer, the shape-retention wire 50 will assume its “preformed” shape. The shape-retention wire 50 is flexible while a clinician negotiates the catheter 22 through the vasculature to reach the heart and enter an atrial chamber. Once the distal end 12 of the catheter 22 reaches the desired cardiac cavity with the ablation electrode section 20, the shape-retention wire 50 assumes a pre-formed shape form, e.g., the C-shaped configuration of the ablation electrode section 20, to accurately orient the ablation electrode section 20 within the cardiac cavity for the procedure to be performed.

As further shown in FIGS. 2 and 3, an array of electrode rings 38 is also provided along the ablation electrode section 20 at the distal end 12 of the catheter 22. Each of the electrode rings 38 is spaced apart equidistant from each adjacent electrode ring 38. However, the electrode rings 38 may be spaced apart at differing regular or irregular intervals depending upon the desired effect of the ablation electrode section 20. Further, the greater or fewer electrode rings 38 may be mounted on the distal end 12 of the catheter 22 than the number depicted, again depending upon the desired effect of the ablation electrode section 20. Each of the electrode rings 38 defines a contoured opening 40, the structure and function of which are further described below. Additionally, as shown in FIG. 3, the catheter 22 may house a wire lumen 46 and a shape-retention wire 50.

FIGS. 4 and 5 depict a portion of the ablation electrode section 20 at the distal end 12 of the catheter 22 in greater detail. The catheter 22 as depicted in FIGS. 4 and 5 is presented in a straight, linear form as opposed to the curved form of FIGS. 2 and 3 for ease of depiction of the structures therein. As previously noted, the distal end 12 of the catheter 22 may be caused to take on any of a number of desired shapes depending upon the intended application of the catheter 22 as further described herein below.

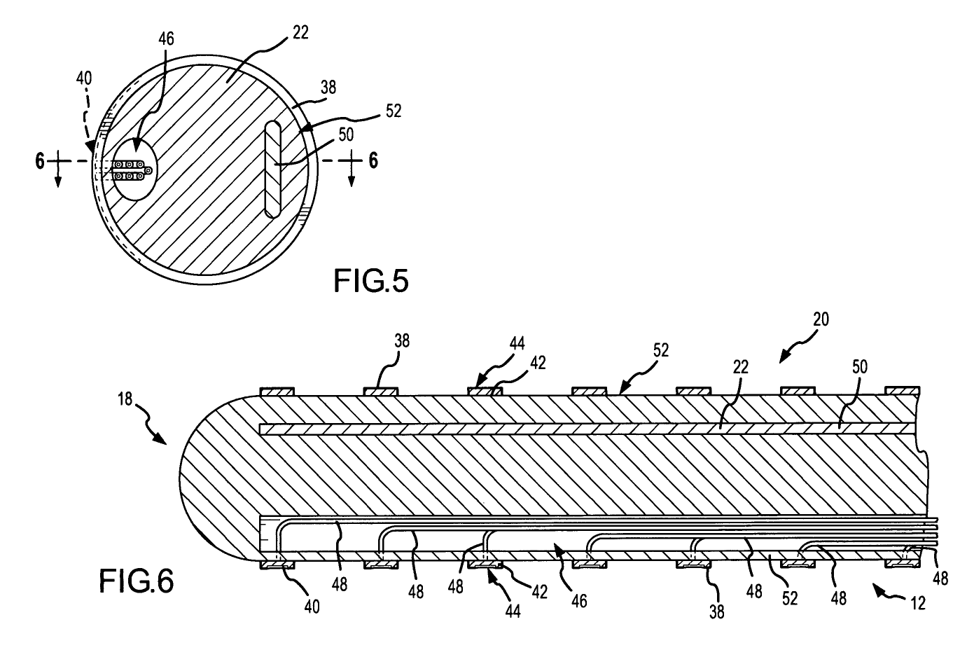

As indicated above, the catheter 22 defines a wire lumen 46 as shown to good advantage in FIGS. 5 and 6. The wire lumen 46 houses a plurality of electrode lead wires 48, which travel from the electrical connector 4 at the proximal end 14 of the catheter assembly 2 to the distal end 12 of the catheter 22. Each of the electrode lead wires 48 may be coupled with a respective electrode ring 38, thereby allowing each electrode ring 38 to be individually addressable. The electrode lead wires 48 transmit radio frequency (RF) energy from an energy generator (not shown) to energize the electrode rings 38. Because each electrode ring 38 is individually addressable, RF energy can be transmitted to only one, several, or all of the electrode rings 38 at a single instant. The electrode rings 38 may be evenly spaced along the ablation electrode section 20 of the catheter 22 in order to create a continuous, linear lesion in the target tissue. Further, RF energy at different power levels can be transmitted to different electrode rings 38. It should be noted that one of the electrode lead wires could also be coupled with several electrode rings to provide for an addressable subset of the electrode rings. Also, a single electrode lead could be coupled with all of the electrode rings as further described below.

Each of the ring electrodes 38 is formed of a conductive band 42 attached circumferentially about the outer surface of the catheter 22. The conductive bands 42 may be composed of platinum, gold, stainless steel, iridium, or alloys of these metals, or other biocompatible, conductive material. The conductive bands 42 of each electrode ring 38 have an electrically insulating, polymer surface coating 44. The surface coating 44 is preferably formed of a material with high dielectric properties that can be applied in a very thin layer. Exemplary surface coatings may include thin coatings of polyester, polyamides, polyimides, and blends of polyurethane and polyimides. An aperture is formed in the surface coating 44 to create a contoured opening 40 that exposes a small area of the conductive band 42. Each contoured opening 40 is preferably positioned circumferentially about the catheter 22 inline with each adjacent contoured opening 40. The contoured openings 40 may extend between about 1/10 and ⅓ the circumference of the ring electrodes 38. Longer countered openings 40 make it easier to position the ablation electrode section 20 adjacent the target tissue. However, longer contoured openings 40 can also lead to greater heat generation and the potential for hot spots as further discussed below. A balance in the length of the contoured openings 440 should thus be struck depending upon the particular application.

A corresponding electrode lead wire 48 is coupled to the conductive band 42 of a respective electrode ring 38, for example, as shown to good advantage in FIG. 6. Each electrode lead wire 48 exits the wire lumen 46, protrudes through the exterior catheter wall 52, and is electrically connected to the conductive band 42 of the ring electrode 38. As depicted in FIG. 6, each electrode lead wire 48 may be coupled to a respective conductive band 42 directly adjacent the contoured opening in the surface coating 44. However, the electrode lead wires 48 may alternately be coupled to the conductive bands 42 at any location along the circumference of the conductive bands 42 as long as the conductive bands 42 are good electrical conductors and good electrical connections are created.

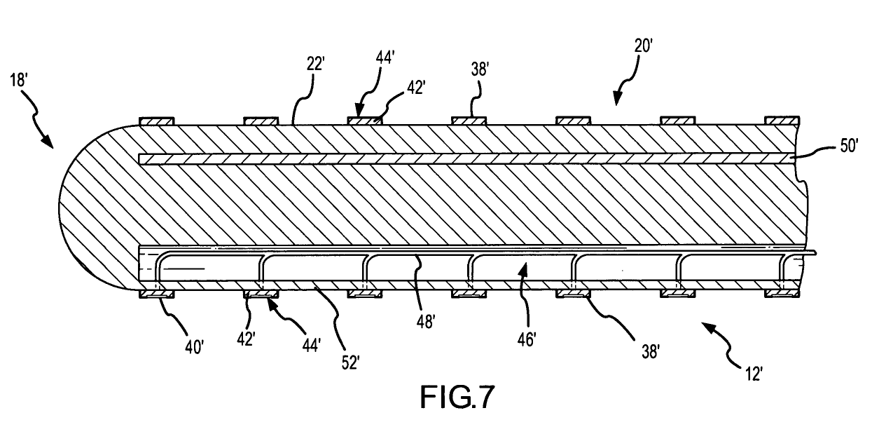

Alternatively, as shown in the embodiment of FIG. 7, a single electrode lead wire 48′ is coupled with each of the conductive bands 42′ of the ring electrodes 38′. The distal end 12′ of the catheter 22′ of this embodiment forms an ablation electrode section 20′ generally identical to the ablation electrode section of the previous embodiment. Each of the ring electrodes 38′ is covered with an insulating surface coating 44′ that defines a contoured opening 40′ exposing a conductive band 42′ underneath. The catheter 22′ may further include a shape memory wire 50′ and a wire lumen 46′ as in the previous embodiment. Only a single electrode lead wire 48′ is housed in the wire lumen 46′ that may have a plurality of branches that attach the electrode lead wire 48′ to each of the electrode rings 38′. As is evident from the depiction in FIG. 7, the ring electrodes 38′ in this embodiment are not individually addressable and each ring electrode 38′ will be simultaneously and generally equally powered upon application of energy through the electrode lead wire 48′ from an energy source.

FIG. 8 depicts a further alternative embodiment of the invention. In this embodiment, a helical electrode wire 38″ is formed of a conductive wire 42″ and covered with an insulating, polymer surface coating 44″ The helical electrode wire 38″ is attached circumferentially about the outer surface of the distal end 12″ of the catheter 22″ along the ablation electrode section 20″. The helical electrode wire 38″ may be the same wire as an electrode lead wire housed within a wire lumen (not shown) in the catheter 22″. In such a design, the electrode lead wire may exit the exterior wall of the catheter 22″, begin wrapping around the exterior surface of the catheter 22″ distally to form the helical electrode wire 38″, and terminate adjacent the distal tip 18″. The conductive wire 42″ may be composed of platinum, gold, stainless steel, iridium, or alloys of these metals, or other biocompatible, conductive material. The polymer surface coating 44″ may be composed of a thin coating of any suitable insulating material, for example, polyester, polyamides, polyimides, and blends of polyurethane and polyimides. The helical electrode wire 38″ may be formed of a standard insulated wire having a metal wire enveloped by an insulating sheathing, rather than specially creating an electrode wire. A plurality of apertures is formed in the surface coating 44″ to create a series of contoured openings 40″ that each expose a small area of the conductive wire 42″. Each contoured opening 40″ is preferably positioned circumferentially about the catheter 22 inline with each adjacent contoured opening 40″, thus forming a linear array parallel to the longitudinal direction of the catheter 22″. By alignment of the contoured openings 40″ and by spacing each turn of the helical electrode wire 38″ sufficiently close to adjacent turns at regular, narrow intervals, sufficient energy overlap should result to produce a linear lesion a in the target tissue.

The purpose of the surface coating on the ring electrodes or along the helical electrode wire is primarily two-fold. First, uninsulated conductive bands or wire electrodes have been demonstrably shown to overheat cardiac tissue along certain points of the ablation electrode section. Such excessive heat can transform the tissue beyond mere necrosis and actually cause undesirable tissue destruction (e.g., charring and endothelial damage) that can compromise the integrity of the myocardium, e.g., through perforation or tamponade, or can lead to embolic events. Some theories suggest that an energized ring electrode or wire electrode exhibits a non-uniform power density that results in such “hot spots” in certain areas on the ring electrode or along the length of the wire electrode. Another, more likely, rationale for formation of hot spots is related to thermodynamic effects exhibited at the interface of the electrodes and the catheter. While the power density in the electrodes remains uniform, heat dissipation in the active ablation area is not because the plastic catheter shaft material is a poor heat conductor and is unable to adequately dissipate the heat from the metal electrode. Thus, localized temperature variations may develop. By coating the metal electrode with an insulator, rather than transferring energy to the surrounding blood or adjacent tissue and thereby creating additional heat, the insulated electrode will act as a heat sink and counter the potential for the formation of hot spots at the edge of the exposed active ablation area.

In order to increase the ability of the electrodes to act as a heat sink, the high dielectric surface coating may be applied in a very thin layer. For example, very thin coatings of polyester, polyamides, polyimides, and blends of polyurethane and polyimides, on the order of 2.5/10,000 inch to 1/1000 inch may be applied to the electrodes. By minimizing the thickness of the polymer surface coating, the thermal insulating effects of the dielectric polymer material is minimized. Thus, increased thermal transfer between the tissue and the insulated portion of the electrode can be achieved to mitigate the formation of hot spots along the edge areas interfacing with the catheter wall.

Second, the electrically insulating surface coating on each of the electrode rings is important to minimize the coagulation of blood in the surrounding cardiac cavity. Uninsulated electrodes create coagulum that often cakes about the conductive band or electrode wire, potentially impacting the efficacy of the ablation electrode section. Of even more concern is the possibility that a large body of coagulum could form on the catheter, break free in the bloodstream, and potentially cause an embolism or stroke. Because the contoured openings only expose a small area of the conductive bands or the electrode wire, the possibility of coagulum formation is minimized. Further, because the contoured openings are positioned and arranged to be in direct contact with the target tissue during the application of RF energy, the likelihood of coagulum formation is again decreased.

The contoured openings may be formed by laser, chemical, or other common etching processes to remove a portion of the surface coating to expose the conductive material underneath. The edges or corners of any of the shapes of the contoured openings may be curved, rounded, or otherwise contoured in order to additionally minimize any edge effects that could arise due to the imposition of a sharp edge or point. The ring electrodes and the helical electrode wire may be between approximately 0.5 mm and 4 mm wide. The contoured openings may correspondingly have dimensions on the order of 25-80% of the width of the conductive bands and extend up to one-third the circumference of the conductive bands.

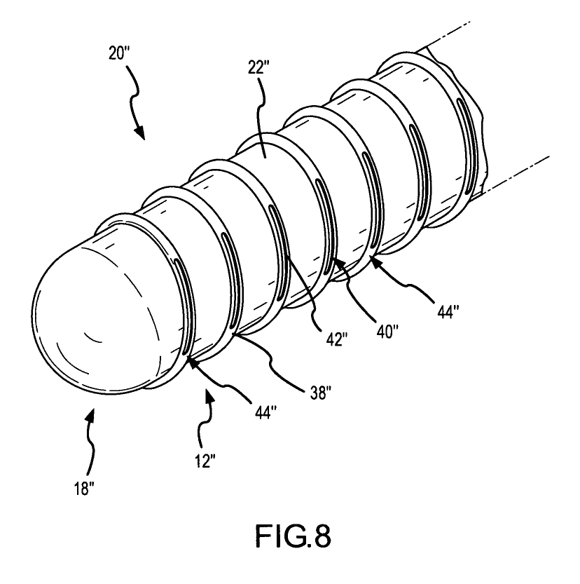

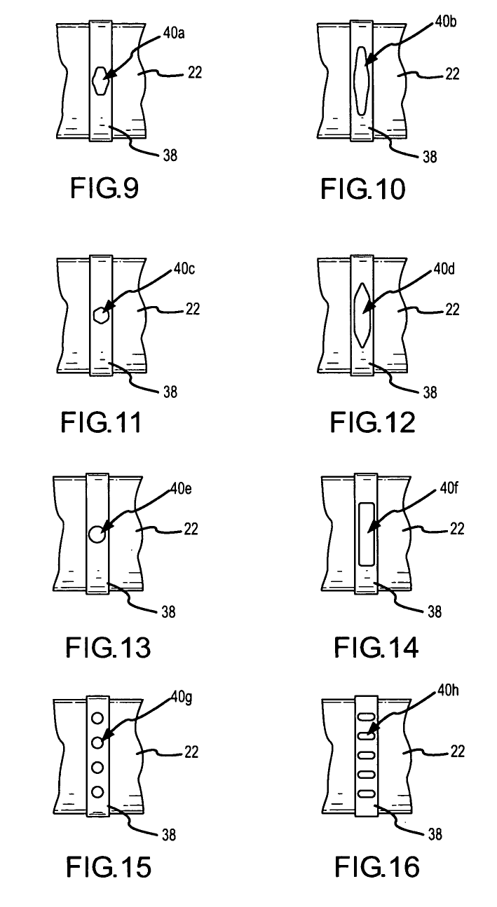

FIGS. 2, and 4 depict one exemplary form of a contoured opening 40 as an elliptical opening in the surface coating 44. FIG. 8 depicts another exemplary form of a contoured opening 40″ as an oval opening in the surface coating 44″. Other exemplary forms for contoured openings according to the present invention are depicted in FIGS. 9-14. FIG. 9 depicts a contoured opening 40a in the surface coating 44 of the ring electrode 38 on the catheter 22 in the form of an elongate, diamond shape with rounded corners. Similar elongate, regular polygonal shapes, with or without rounded edges or corners, are also contemplated by the present invention. FIG. 10 depicts a contoured opening 40b in the surface coating 44 of the ring electrode 38 on the catheter 22 in the form of an elongated, symmetrical curvilinear shape oriented parallel to the circumference of the ring electrode 38. The present invention contemplates the formation of other symmetrical and asymmetrical curvilinear shapes. FIG. 11 depicts a contoured opening 40c in the surface coating 44 of the ring electrode 38 on the catheter 22 in the form of a hexagon with rounded corners. FIG. 12 depicts a contoured opening 40d in the surface coating 44 of the ring electrode 38 on the catheter 22 in the form of an elongated hexagonal shape oriented parallel to the circumference of the ring electrode 38. Similar polygonal shapes with or without rounded edges or corners, for example, a square, a pentagon, or an irregular polygon, are also contemplated by the present invention. FIG. 13 depicts a contoured opening 40e in the surface coating 44 of the ring electrode 38 on the catheter 22 in the form of a circle. FIG. 14 depicts a contoured opening 40f in the surface coating 44 of the ring electrode 38 on the catheter 22 in the form of an long, rectangular shape with rounded corners oriented parallel to the circumference of the ring electrode 38. FIG. 15 depicts an array of contoured openings 40g in the surface coating 44 of the ring electrode 38 on the catheter 22 in the form of circles extending along a length of the ring electrode 38. FIG. 16 depicts an array of contoured openings 40h in the surface coating 44 of the ring electrode 38 on the catheter 22 in the form of ovals extending along a length of the ring electrode 38. It should be apparent that arrays of contoured openings similar to those depicted in FIGS. 15 and 16 could be of any shape and could be of mixed shapes.

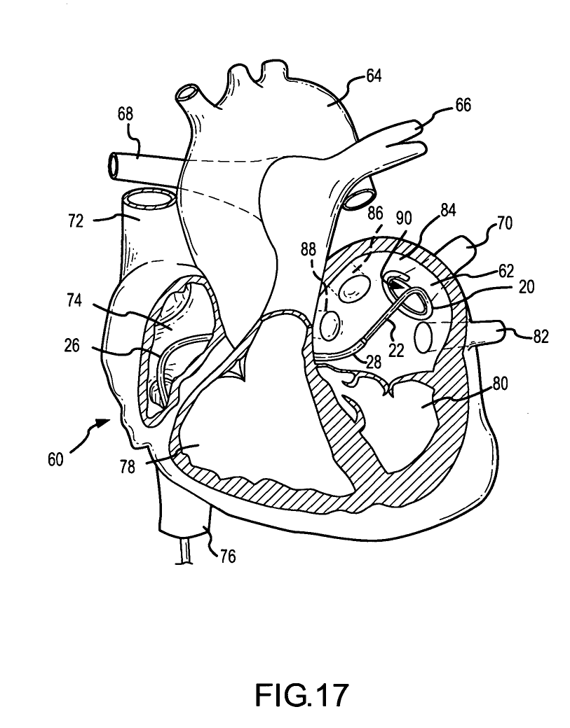

FIG. 17 schematically depicts the catheter 22 and ablation electrode section 20 according to a generic ring electrode embodiment of the present invention being used to ablate tissue in a left superior pulmonary vein 70. FIG. 17 includes a number of primary components of the heart 60 to orient the reader. In particular, starting in the upper left-hand portion of FIG. 17, and working around the periphery of the heart 60 in a counterclockwise fashion, the following parts of the heart 60 are depicted: the superior vena cava 72, the right atrium 74, the inferior vena cava 76, the right ventricle 78, the left ventricle 80, the left inferior pulmonary vein 82, left superior pulmonary vein 70, the left atrium 84, the right superior pulmonary vein 86, the right inferior pulmonary vein 88, the left pulmonary artery 66, the arch of the aorta 64, and the right pulmonary artery 68.

The distal end of the ablation electrode section 20 is positioned adjacent to the ostium 90 of the left superior pulmonary vein 70 using known procedures. For example, to place the ablation electrode section 20 in the position shown in FIG. 17, the right venous system may be first accessed using the “Seldinger technique.” In this technique, a peripheral vein (such as a femoral vein) is first punctured with a needle and the puncture wound is dilated with a dilator to a size sufficient to accommodate an introducer, e.g., the outer guiding introducer 26. The outer guiding introducer 26 with at least one hemostatic valve is seated within the dilated puncture wound while maintaining relative hemostasis. From there, the outer guiding introducer 26 is advanced along the peripheral vein, into the inferior vena cava 76, and into the right atrium 74. A transeptal sheath may be further advanced through the outer guiding introducer 26 to create a hole in the interatrial septum between the right atrium 74 and the left atrium 84.

Once the outer guiding introducer 26 is in place in the right atrium 74, the inner guiding introducer 28, housing the catheter 22 with the ablation electrode section 20 on the distal end, is introduced through the hemostatic valve of the outer guiding introducer 26 and navigated into the right atrium 74, through the hole in the interatrial septum, and into the left atrium 84. Once the inner guiding introducer 28 is in the left atrium 84, the ablation electrode section 20 of the catheter 22 and may be advanced through the distal tip of the inner guiding introducer 28. The ablation electrode section 20 as shown in FIG. 17 is being inserted into the ostium 90 of the left superior pulmonary vein 70 to contact the tissue of the walls of the vein. The configuration of the ablation electrode section 20, for example, in a shape as depicted in FIGS. 2 and 3, is advantageous for maintaining consistent contact with tissue in a generally cylindrical vessel. Other configurations of the ablation electrode section 20 may be used to greater advantage on tissue surfaces of other shapes.

While the ablation electrode 20 is in the left superior pulmonary vein 70, the ablation electrode section 20 may be energized to create the desired lesion in the left superior pulmonary vein 70. The RF energy emanating from the ablation electrode section 20 is transmitted through the portions of the conductive bands exposed through the contoured openings. The contoured openings are placed in contact with the tissue, for example, by employing one or more of the orientation structures described above within the catheter 22. Thus, a lesion is formed in the tissue by the RF energy. In order to form a sufficient lesion, it is desirable to raise the temperature of the tissue to at least 50° C. for an appropriate length of time (e.g., one minute). Thus, sufficient RF energy must be supplied to the electrode to produce this lesion-forming temperature in the adjacent tissue for the desired duration.

Although various embodiments of this invention have been described above with a certain degree of particularity, or with reference to one or more individual embodiments, those skilled in the art could make numerous alterations to the disclosed embodiments without departing from the spirit or scope of this invention. It is intended that all matter contained in the above description and shown in the accompanying drawings shall be interpreted as illustrative only of particular embodiments and not limiting. All directional references (e.g., proximal, distal, upper, lower, upward, downward, left, right, lateral, front, back, top, bottom, above, below, vertical, horizontal, clockwise, and counterclockwise) are only used for identification purposes to aid the reader's understanding of the present invention, and do not create limitations, particularly as to the position, orientation, or use of the invention. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the basic elements of the invention as defined in the following claims.

What is claimed is:

1. A catheter comprising
an elongate shaft defining a lumen;
a proximal section;
at least one electrode positioned about a distal end of the elongate shaft, wherein the at least one electrode further comprises
a conductive material; and
an insulating coating substantially covering the conductive material, wherein the insulating coating defines a contoured opening that exposes an area of the conductive material; and
at least one electrode lead housed within the lumen, extending from the proximal section, and coupled at a distal end with the at least one electrode.

2. The catheter of claim 1, wherein the at least one electrode comprises a ring electrode that encircles a portion of the elongate shaft.

3. The catheter of claim 1, wherein the at least one electrode comprises a plurality of ring electrodes, wherein each of the plurality of ring electrodes encircles a respective portion of the elongate shaft and is spaced apart from each adjacent ring electrode by a uniform distance.

4. The catheter of claim 3, wherein the contoured openings of each of the plurality of ring electrodes are arranged longitudinally along the distal end of the elongate shaft in a linear array.

5. The catheter of claim 1, wherein the at least one electrode lead couples with the conductive material of the at least one electrode.

6. The catheter of claim 3, wherein
the at least one electrode lead comprises a plurality of electrode leads; and
each of the plurality of electrode leads couples with the conductive material of a respective one of the plurality of electrode rings.

7. The catheter of claim 3, wherein
the at least one electrode lead comprises a plurality of electrode leads; and
each of the plurality of electrode leads couples with the conductive material of a subset of the plurality of ring electrodes.

8. The catheter of claim 1, wherein the at least one electrode comprises a helical wire electrode wrapped around a section of the distal end of the elongate shaft.

9. The catheter of claim 8, wherein
the helical wire electrode comprises an insulated wire composed of a metal wire enclosed within an insulating sheathing;
the conductive material comprises the metal wire; and
the insulating coating comprises the insulating sheathing.

10. The catheter of claim 8, wherein the contoured opening further comprises a plurality of contoured openings spaced apart along a length of the helical wire electrode.

11. The catheter of claim 10, wherein each of the plurality of contoured openings is positioned circumferentially about the elongate shaft in-line with each adjacent contoured opening to form a linear array parallel to the longitude of the elongate shaft.

12. The catheter of claim 10, wherein each turn of the helical electrode wire is spaced sufficiently close to each adjacent turn at a regular, narrow interval to provide sufficient energy overlap to produce a linear lesion correlative to a length of the helical wire electrode.

13. The catheter of claim 1, wherein the contoured opening is formed as a shape selected from a group of shapes consisting of a circle, an oval, a symmetrical curvilinear shape, an asymmetric curvilinear shape, a diamond, a square, a rectangle, a hexagon, and a polygon.

14. The catheter of claim 1, wherein the contoured opening comprises an array of contoured openings along a length of the at least one electrode.

15. The catheter of claim 1, wherein the contoured opening extends between 25% and 80% of a width of the at least one electrode.

16. The catheter of claim 1, wherein the contoured opening extends between 1/10 and ⅓ of a circumference of the shaft.

17. A catheter comprising
an elongate shaft defining a lumen;
a proximal section;
a plurality of ring electrodes positioned about a distal end of the elongate shaft,
wherein each of the plurality of ring electrodes encircles a respective portion of the elongate shaft and is spaced apart from each adjacent ring electrode by a uniform distance; and
wherein each of the plurality of ring electrodes further comprises
a conductive material; and
an insulating coating substantially covering the conductive material,
wherein the insulating coating defines a contoured opening that exposes an area of the conductive material, and
wherein the contoured openings of each of the plurality of ring electrodes are arranged longitudinally along the distal end of the elongate shaft to form a linear array; and
at least one electrode lead housed within the lumen, extending from the proximal section, and coupled at a distal end with the plurality of ring electrodes.

18. A catheter comprising
an elongate shaft defining a lumen;
a proximal section;
a helical wire electrode wrapped about a distal end of the elongate shaft, wherein the helical wire electrode further comprises
a conductive material; and
an insulating coating substantially covering the conductive material, wherein the insulating coating defines a plurality of contoured openings that each expose an area of the conductive material, wherein
each of the plurality of contoured openings is positioned circumferentially about the elongate shaft in-line with each adjacent contoured opening to form a linear array parallel to the longitude of the elongate shaft, and
each turn of the helical electrode wire is spaced sufficiently close to each adjacent turn at a regular, narrow interval to provide sufficient energy overlap to produce a linear lesion correlative to a length of the helical wire electrode; and
at least one electrode lead housed within the lumen, extending from the proximal section, and coupled at a distal end with the helical electrode wire.

19. An electrode for use in conjunction with a cardiac ablation catheter, the electrode comprising
a conductive band sized to encircle an outer surface of the catheter;
an insulating coating substantially covering an outer surface of the conductive band,
wherein the insulating coating defines a contoured aperture exposing a portion of the conductive band; and
a lead wire electrically coupled with the conductive band.

20. The catheter of claim 19, wherein the lead wire couples with the conductive band at a point adjoining the contoured aperture.

21. The sensor of claim 19, wherein the conductive band comprises a conductive material selected from the group consisting of platinum, gold, stainless steel, iridium, and alloys of these metals.

22. The sensor of claim 19, wherein the insulating coating is applied in a very thin layer to function as a poor thermal insulator.

23. The sensor of claim 19, wherein the contoured opening extends between 25% and 80% of a width of the at least one electrode.

24. The sensor of claim 19, wherein the contoured opening extends between 1/10 and ⅓ of a circumference of the shaft.

25. A method for minimizing variations in power density in a surface electrode positioned on a catheter, the method comprising
coating a conductive material portion of the surface electrode with a biocompatible, electrically insulating coating; and
forming a contoured aperture within the electrically insulating coating to expose an area of the conductive material portion.
